(19) United States
(12) Patent Application Publication
(10) Pub. No.: US 2008/0208002 A1
(43) Pub. Date: Aug. 28, 2008

(54) ENDOSCOPE

(75) Inventor: Koji MARUYAMA, Hachioji-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/037,523

(22) Filed: Feb. 26, 2008

(30) Foreign Application Priority Data
Feb. 28, 2007 (JP) 2007-050631

(51) Int. Cl. A61B 1/005

(52) U.S. Cl. 600/131

(57) ABSTRACT

An endoscope includes a thin and long flexible insertion portion extended from an operation portion having a grasping portion. Further, the endoscope is configured such that the operation portion includes a holding portion for holding a distal end portion, a bending portion, or the like, which are distal end side portions of the insertion portion, so as to thereby prevent a trouble which may be caused by an article being placed on the insertion portion, or by the insertion portion being stepped by the user, or the like.

### BACKGROUND OF THE INVENTION

1. Field of the Invention

The present invention relates to an endoscope having an insertion portion extended from an operation portion.

2. Description of the Related Art

Endoscopes have been widely used in medical fields and industrial fields. An endoscope used in a medical field is capable of observing an organ in a body cavity of a subject by inserting a thin and long insertion portion into the body cavity, and further performing various medical treatment or inspection by inserting a treatment instrument into the body cavity through an insertion channel provided in the insertion portion, as required.

On the other hand, an endoscope used in an industrial field is capable of performing observation of the presence or absence of damage, corrosion, and the like, in parts to be inspected, or performing various repair by inserting a thin and long insertion portion of the endoscope into the inside of a jet engine, a pipe in a plant, and the like.

An industrial endoscope apparatus is configured, for example, by an endoscope which has a thin and long insertion portion provided with, at a distal end portion thereof, an image pickup unit having an image pickup devices such as an image pickup lens and a CCD, and having a light emitting device such as an LED, and by an apparatus main body to which the endoscope is connected.

For example, in Japanese Patent Laid-Open No. 2006-142039, there is disclosed an endoscope apparatus which is capable of reducing the load to the arm of a user grasping the endoscope, and enabling a twisting operation of the insertion portion and a bending operation of a bending portion to be freely performed, and which is hence excellent in grasping property and operability.

In the endoscope apparatus disclosed in Japanese Patent Laid-Open No. 2006-142039, the endoscope can be handled by the user in the state where the apparatus main body 4A is attached to the user's body as shown in FIG. 19 of Japanese Patent Laid-Open No. 2006-142039, or in the state where the apparatus main body 4A is placed on the surface of a work desk 90, and the like, as shown in FIG. 20 of Japanese Patent Laid-Open No. 2006-142039.

### SUMMARY OF THE INVENTION

According to the present invention, there is provided an endoscope which includes a thin and long flexible insertion portion extended from an operation portion having a grasping portion, and in which a holding portion for holding a distal end side portion of the insertion portion is provided in the operation portion.

### BRIEF DESCRIPTION OF THE DRAWINGS

FIG. 1 is a view for explaining an endoscope apparatus;

FIG. 2 is a view for explaining a configuration of an operation portion of an endoscope;

FIG. 3 is a front view of the operation portion;

FIG. 4 is a view for explaining configuration example of a holding member;

FIG. 5 is a view for explaining effects of the endoscope having a holding portion in the operation portion;

FIG. 6 is a view for explaining a holding member provided with a square groove as another configuration example of the holding portion;

FIG. 7 is a view for explaining a holding member provided with a V-shaped groove as another configuration example of the holding portion;

FIG. 8 is a view for explaining a holding member provided with three square grooves;

FIG. 9 is a side view for explaining the operation portion to which a distal end holding member is attached;

FIG. 10 is a side view showing a form in which the endoscope apparatus is carried in the state where the operation portion is attached to the apparatus main body, and where the insertion portion is wound and held;

FIG. 11 is a perspective view showing a form in which the endoscope apparatus is carried in the state where the operation portion is attached to the apparatus main body, and where the insertion portion is wound and held;

FIG. 12 is a plan view for explaining the holding portion in which the insertion portion is wound;

FIG. 13 is a plan view for explaining the holding portion in which the insertion portion is wound;

FIG. 14 is a perspective view for explaining the operation portion to which a three point supporting mechanism is mounted;

FIG. 15 is a perspective view for explaining a state where a reel housing member is held in the operation portion;

FIG. 16 is a perspective view for explaining a state where a pocket shaped housing portion is held in the operation portion;

FIG. 17 is a view for explaining a bending lock knob provided in the operation portion;

FIG. 18 is a view for explaining effects of a notifying portion provided in the bending lock knob; and

FIG. 19 is a view for explaining a grasping portion proximal end portion which configures a grasping portion.

### DETAILED DESCRIPTION OF THE PREFERRED EMBODIMENTS

In the following, embodiments according to the present invention will be described with reference to the accompanying drawings.

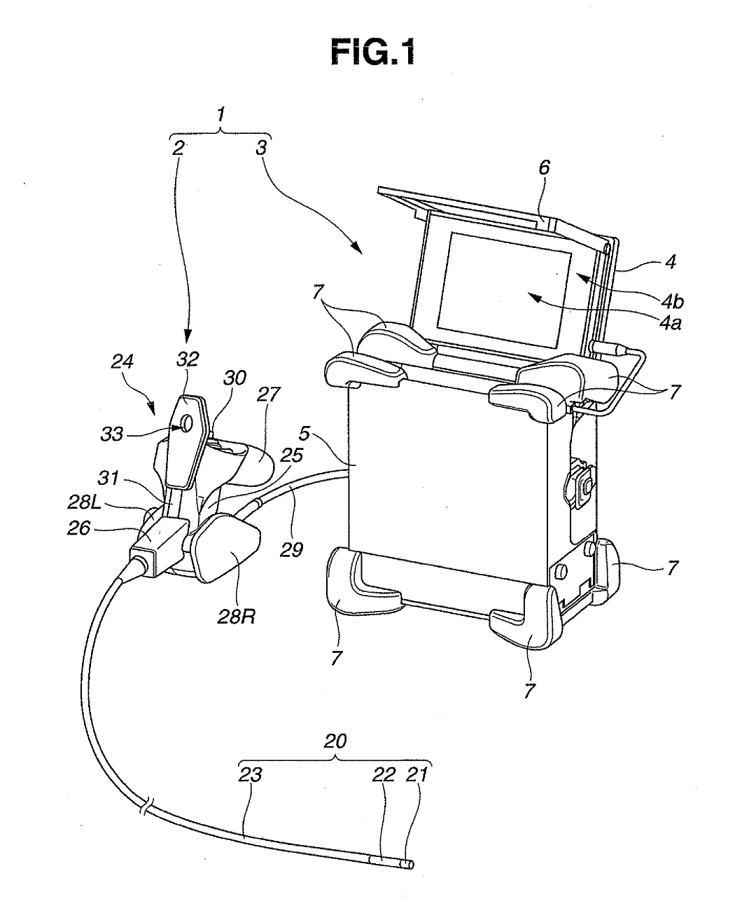

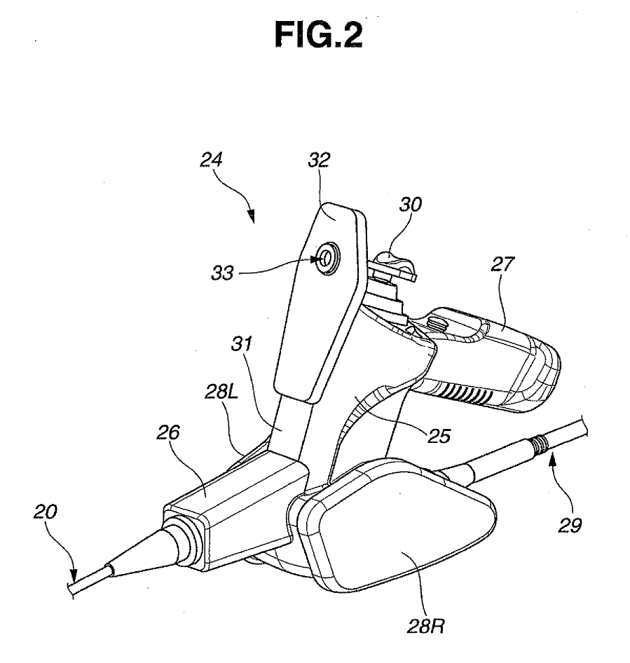

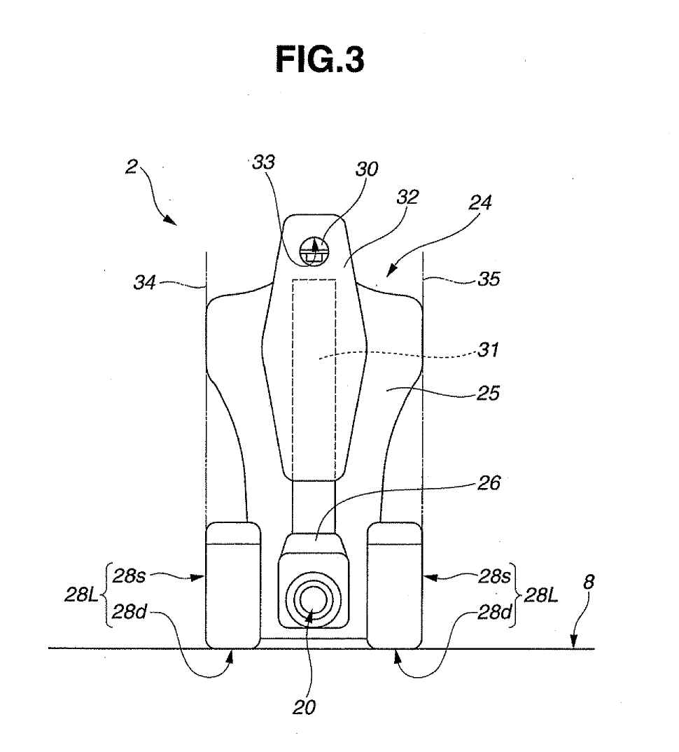

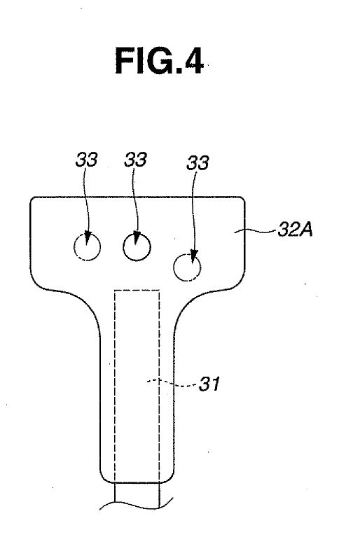

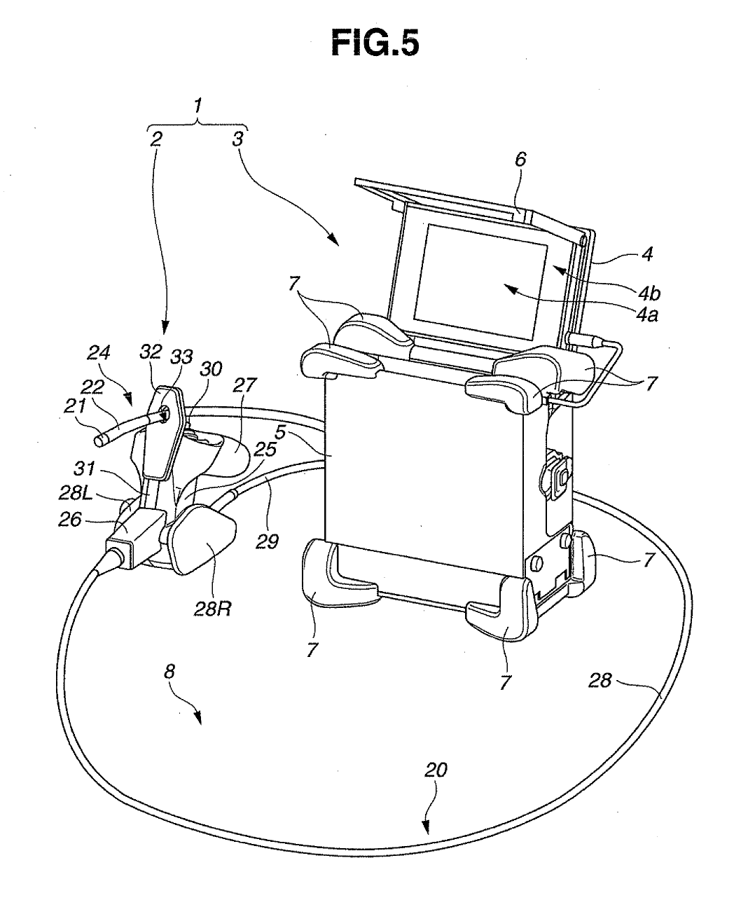

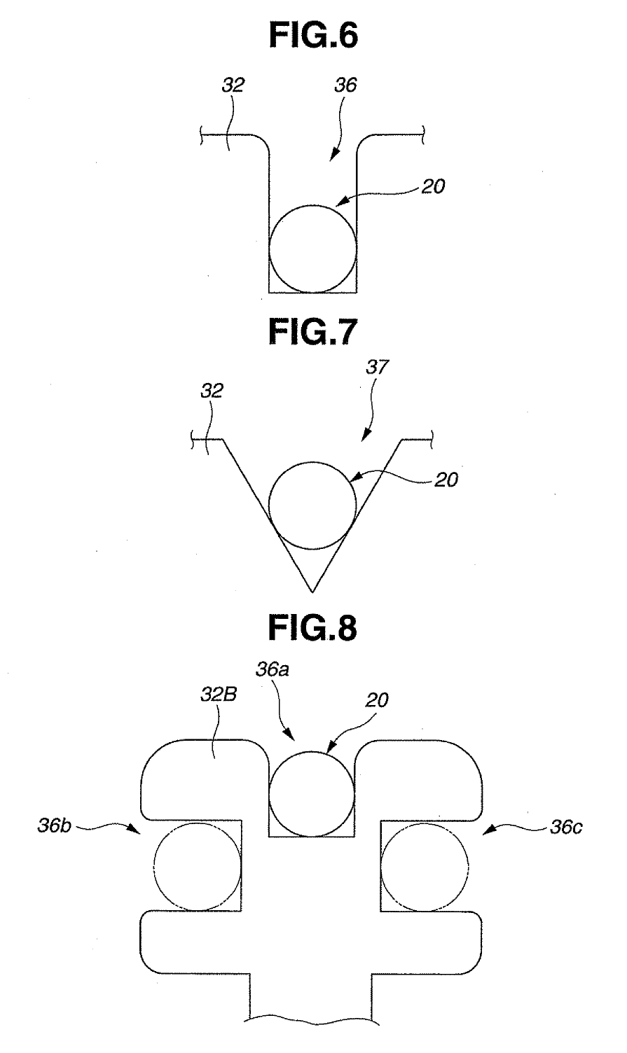

FIG. 1 to FIG. 8 relate to an embodiment according to the present invention. FIG. 1 is a view for explaining an endoscope apparatus configured by an endoscope and an apparatus main body provided with a monitor. FIG. 2 is a perspective view for explaining a configuration of an operation portion of the endoscope. FIG. 3 is a front view of the operation portion. FIG. 4 is a view for explaining configuration example of a holding member. FIG. 5 is a view for explaining effects of the endoscope having a holding portion in the operation portion. FIG. 6 is a view for explaining a holding member provided with a square groove as another configuration example of the holding portion. FIG. 7 is a view for explaining a holding member provided with a V-shaped groove as another configuration example of the holding portion. FIG. 8 is a view for explaining a holding member provided with three square grooves.

As shown in FIG. 1, an endoscope apparatus 1 is configured by including an endoscope 2 and an apparatus main body 3 to which the endoscope 2 is electrically connected.

The endoscope 2 is an industrial endoscope in which an optical lens, an image pickup device (not shown), an optical device, a light emitting device, and the like, are incorporated in a distal end portion 21 of an insertion portion 20. The apparatus main body 3 is configured by including an image processing portion which drives the image pickup device and generates a video signal from an image signal outputted from the image pickup device, a monitor 4 which is a display device for receiving the video signal outputted from the image processing portion and displaying an endoscopic image, and a battery (not shown) which supplies electric power to the optical device and the monitor 4, and the like.

The apparatus main body 3, for example, has a box-like shape, and the monitor 4 is fixed to an exterior housing 5 configuring the apparatus main body 3, via a hinge (not shown) or the like. In the present embodiment, the monitor 4 can be freely opened and closed, and is configured such that in the closed state, the rear face of a monitor face 4b having an image display surface 4a is arranged on the rear face side of the exterior housing 5 in FIG. 1.

Reference numeral 6 denotes a cover which protects the image display surface 4a by covering the image display surface 4a when the endoscope apparatus 1 is not used. Reference numeral 7 denotes a leg portion, which is formed of, for example, rubber such as NBR, and attached to the corner portion of the exterior housing 5. For example, when the leg portions 7 are placed on the ground surface, the floor surface, or the like, the apparatus main body 3 is capable of being in a vertical disposition in which the image display surface 4a of the monitor 4 in the closed state is arranged substantially vertical to the ground surface, or in a horizontal disposition in which the image display surface 4a of the monitor 4 in the closed state is arranged substantially in parallel with the ground surface.

The endoscope 2 is configured by including the thin and long flexible insertion portion 20, an operation portion 24 successively provided to a proximal end portion of the insertion portion 20, and a flexible universal cord 29 extended from the operation portion 24. The insertion portion 20 is configured by successively providing, from the distal end side, the distal end portion 21, a bending portion 22 freely bendable, for example, in the up, down, left and right directions, and a flexible tube portion 23 having flexibility. An electrical cable (not shown) extended from the apparatus main body 3 to the light emitting device in the distal end portion 21, a signal cable (not shown) for transmitting a drive control signal of the image pickup device or an image signal subjected to photoelectric conversion by the image pickup device, and the like, are inserted into the universal cord 29.

As shown in FIG. 1 and FIG. 2, the operation portion 24 is configured by including an operation portion main body 25, an operation wire arrangement portion 26, a grasping portion 27, and a side portion configuring portions 28L and 28R. A bending operation instruction lever (hereinafter abbreviated as a bending lever or an operation lever) 30 which is an operation instruction mechanism for effecting a bending operation of the bending portion 22, is provided on the top surface (also referred to as the upper surface) of the operation portion main body 25.

By performing a tilting operation of the bending lever 30 for changing the tilting direction and the tilting angle of the bending lever 30, a traction member (not shown) which is inserted into the insertion portion 20 and in the operation portion 24, is moved so as to make the bending portion 22 bend in a desired direction by a desired amount. The bending portion 22 is configured so as to be in a substantially linear state, when the bending lever 30 is in an upright state.

The operation wire arrangement portion 26 is configured to project from the front lower surface side of the operation portion main body 25. The insertion portion 20 is extended from the distal end surface of the operation wire arrangement portion 26. The longitudinal axis of the operation wire arrangement portion 26 and the insertion axis of the insertion portion 20 are substantially coaxial to each other, in other words, substantially coincide with each other. On the other hand, the grasping portion 27 is configured to project from the rear upper surface side of the operation portion main body 25. The longitudinal axis of the grasping portion 27 is different from the insertion axis of the insertion portion 20. In other words, the longitudinal axis of the grasping portion 27 and the insertion axis of the insertion portion 20 do not coincide with each other, that is, are configured as axes different from each other.

In the present embodiment, the bending lever 30 is placed in the distal end side of the grasping portion 27. The universal cord 29 is extended from the rear lower surface side of the operation portion main body 25.

The side portion configuring portion 28L is provided in the left side surface portion of the operation portion main body 25, when the front of the front portion 31 of the operation portion main body 25 configuring the operation portion 24 is viewed from the side of the insertion portion 20. On the other hand, the side portion configuring portion 28R is provided in the right side surface portion of the operation portion main body 25. When the lower surfaces 28d of the side portion configuring portions 28L and 28R are set, for example, as surfaces in contact with a floor surface 8 as shown in FIG. 3, the operation portion 24 is in the vertical disposition in which the bending lever 30 is substantially orthogonal to the floor surface 8.

On the other hand, when a side surface 28s of the side portion configuring portion 28L or a side surface 28s of the side portion configuring portion 28R is set as a surface in contact with the floor surface 8, the operation portion 24 is in the horizontal disposition in which the bending lever 30 is substantially in parallel with the floor surface 8.

A holding member 32 having a holding portion for holding the insertion portion 20 is provided in the front portion 31 of the operation portion main body 25. The holding member 32 is configured as a separate body from the operation portion main body 25, and is integrally fixed to the operation portion main body 25 by, for example, an adhesive, a screw, or the like. In the fixed state, the upper end side of the holding member 32, which is the upper side in FIG. 3, is configured so as to project by a predetermined amount from the top surface of the operation portion main body 25, and also serves as a guard member for protecting the bending lever 30.

A holding hole 33 serving as the holding portion is formed in the center of an upper end side portion of the holding member 32 projecting from the top surface of the operation portion main body 25. The inside diameter of the holding hole 33 is formed larger by a predetermined dimension than the outer diameter of the insertion portion 20. Thus, when the distal end portion 21, the bending portion 22, or the distal end side of the flexible tube portion 23, which are the distal end side portion of the insertion portion 20, is inserted into the holding hole 33, the insertion portion 20 is held by the holding member 32, and the distal end portion 21 of the insertion portion 20 is arranged in a space.

Note that in the present embodiment, the holding hole 33 is formed in the center of the upper end side portion of the holding member 32. However, the forming position of the holding hole 33 is not limited to the center of the upper end side portion of the holding member 32. For example, the forming position of the holding hole 33 may be a position which when the operation portion 24 is horizontally placed, is arranged, as shown by a two-dot chain line in FIG. 3, between a first abutting line 34 configured by the side surface 28s of the side portion configuring portion 28L and an upper left side surface of the operation portion main body 25, and a second abutting line 35 configured by the side surface 28s of the side portion configuring portion 28R and the upper right side surface of the operation portion main body 25, and which is separated from the outer surface of the operation portion main body 25 by a predetermined distance.

For this reason, the shape of the holding member 32 is also not limited to the shape shown in the figure, and for example, may be substantially T-shape, or the like, as shown in FIG. 4. Note that in the present embodiment, the holding member 32 having the holding portion is configured as a separate body from the operation portion main body 25, but the projecting portion may be formed integrally with the operation portion main body 25. That is, the endoscope 2 includes the thin and long flexible insertion portion 20, the operation portion 24 having the grasping portion 27 from which the insertion portion 20 is extended, the holding hole 33 serving as the holding portion for holding the distal end portion 21 or the bending portion 22, which are the distal end side portion of the insertion portion 20 provided in the operation portion 24, and the like. Note that here, “to be provided in the operation portion 24” means “to be arranged in the operation portion 24”, or “to be formed by modifying a part of the operation portion 24”.

There will be described the effects of the endoscope 2 configured as described above.

When a user once interrupts an endoscopic examination being performed by using the endoscope 2 of the endoscope apparatus 1 according to the present embodiment, the user vertically places the endoscope apparatus 1, that is, the apparatus main body 3 and the endoscope 2, for example, on the floor surface 8. Thereafter, the user inserts the distal end portion 21 or the bending portion 22 of the insertion portion 20 configuring the endoscope 2, into the holding hole 33 of the holding member 32, for example, from the side of the bending lever 30.

Then, as shown in FIG. 5, the insertion portion 20 is held by the holding member 32, without giving a stress to the insertion portion 20. At this time, the distal end portion 21 and the bending portion 22 of the insertion portion 20 are arranged near the operation portion 24, and in a space away from the floor surface 8. This eliminates a trouble that an article is accidentally placed on the distal end portion 21 or the bending portion 22, and a trouble that the distal end portion 21 or the bending portion 22 is stepped on or kicked by user's shoes, or the like.

Further, the operation portion 24 is arranged near the apparatus main body 3, so as to thereby make the insertion portion 20 arranged near the apparatus main body 3 over the whole length of the insertion portion 20. This prevents a trouble that the flexible tube portion 23 configuring the insertion portion 20 is stepped on or kicked by user's shoes, or the like.

Further, when the endoscope apparatus 1 is carried during the work, the distal end side of the flexible tube portion 23 is inserted into the holding hole 33. Thereby, the distal end portion 21 of the insertion portion 20 is arranged close to the user. This prevents a trouble that the distal end portion 21 collides with the floor, a wall, and the like, while the endoscope apparatus 1 is moved.

In this way, the holding portion for holding the distal end side portion of the insertion portion is provided in the operation portion, and the insertion portion is inserted and arranged into the holding portion. Thereby, it is possible to prevent, when the operation portion is placed on the floor, or the like, the distal end portion and the bending portion, which configure the insertion portion, from being laid on the floor surface, and to prevent a trouble that the distal end portion and the bending portion are damaged by being accidentally subjected to a load.

That is, when the work by the endoscope is once interrupted, the endoscope apparatus is left by the user on the work desk, the ground surface, the floor surface, or the like. That is, the apparatus main body and the endoscope which configure the endoscope apparatus are placed on the work desk, the floor surface, or the like. However, even when the apparatus main body and the endoscope are placed on the work desk, there is no possibility that articles, such as a tool, are accidentally placed on the insertion portion or the universal cord. On the other hand, even when the apparatus main body and the endoscope are placed on the floor surface, or the like, there is no possibility that a tool is accidentally placed on the insertion portion or the universal cord. Also, there is no possibility that the insertion portion or the universal cord is accidentally stepped on or kicked by the user, or the like.

Note that in the present embodiment, the holding portion is configured as the holding hole, the holding portion is not limited to a hole. For example, the holding portion may be configured such that a square groove 36, in which the insertion portion 20 can be arranged, is formed in the holding member 32 as shown in FIG. 6. On the opening side of the square groove 36, there are provided chamfers configured by a curved surface or an inclined surface, for preventing the surface of the insertion portion 20 from being damaged. Further, the holding portion may be configured such that a V-shaped groove 37, in which the insertion portion 20 can be arranged, is formed in the holding member 32 as shown in FIG. 7. Further, the holding portion may be configured such that, for example, square grooves 36a, 36b, and 36c, in which the insertion portion 20 can be arranged, are respectively formed in the upper side, the left side, and the right side of the holding member 32B as shown in FIG. 8. In the configuration, when the operation portion 24 is vertically placed, the insertion portion 20 is arranged in the groove 36a, while when the operation portion 24 is horizontally placed, the insertion portion 24 is arranged in one of the groove 36b and the groove 36c. Further, the groove configuring the holding portion is not limited to the square groove and the V-shaped groove. The holding portion may also be configured by recessed portions, such as a U-shaped groove and a semicircular groove.

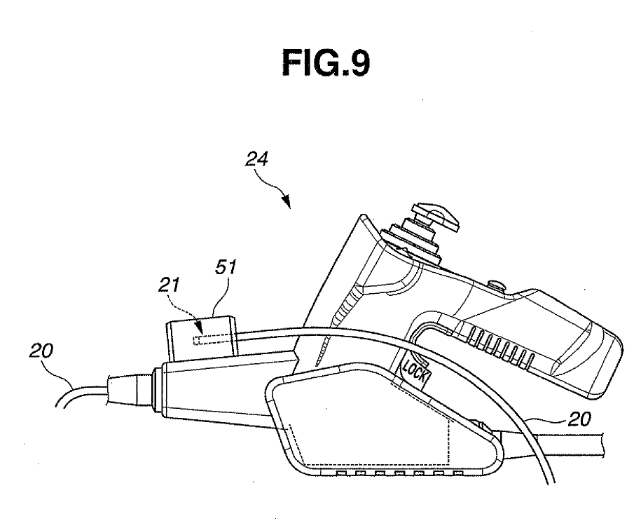

Further, as in an endoscope of another embodiment shown in FIG. 9, it is also possible to use, as the holding portion of the distal end portion 21 of the insertion portion 20, a holding member 51 formed by changing a shape of a part of the operation portion 24, and configured to have a holding portion which is a bottomed hole, and into which the distal end portion 21 of the insertion portion 20 can be inserted. Since the holding member 51 not only holds the distal end portion side of the insertion portion 20, but also houses and protects the distal end portion 21, the holding member 51 highly effectively prevents a trouble that the optical lens, and the like, incorporated in the distal end portion 21 is damaged.

Further, the holding portion of the distal end side portion of the insertion portion 20 is not limited to the configuration in which the holding member 51 is provided in the operation portion 24 as shown in FIG. 9. For example, the holding portion may be configured such that the distal end portion 21 is inserted into a part of the operation portion main body 25 or the apparatus main body 3, or that a shape of the operation portion main body 25 or the apparatus main body 3 is changed into a shape in which the distal end portion 21 can be held. In the holding portion configured by changing a shape of a part of the operation portion main body 25 or the apparatus main body 3, a dedicated holding member need not be provided only for holding the distal end side portion of the insertion portion 20. Further, there is no possibility that the holding member is detached from the endoscope apparatus 1, so as to be lost.

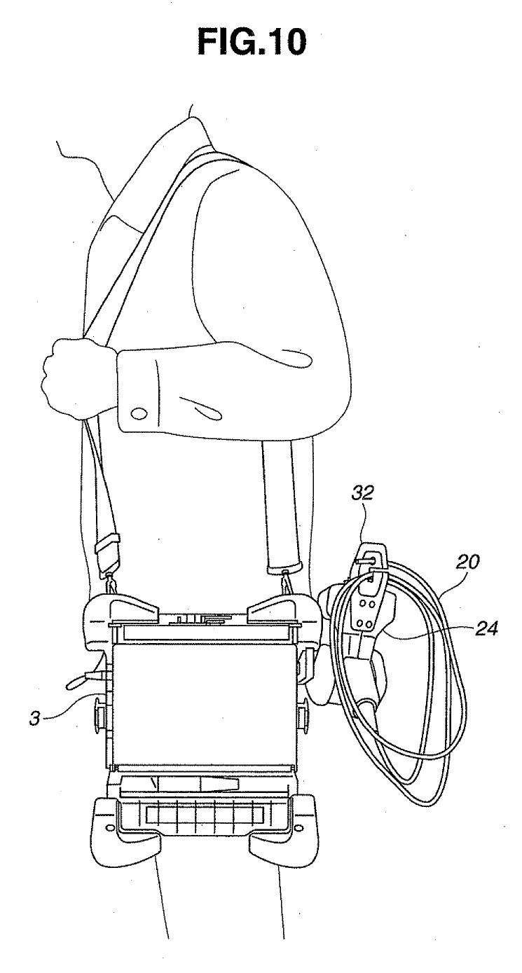

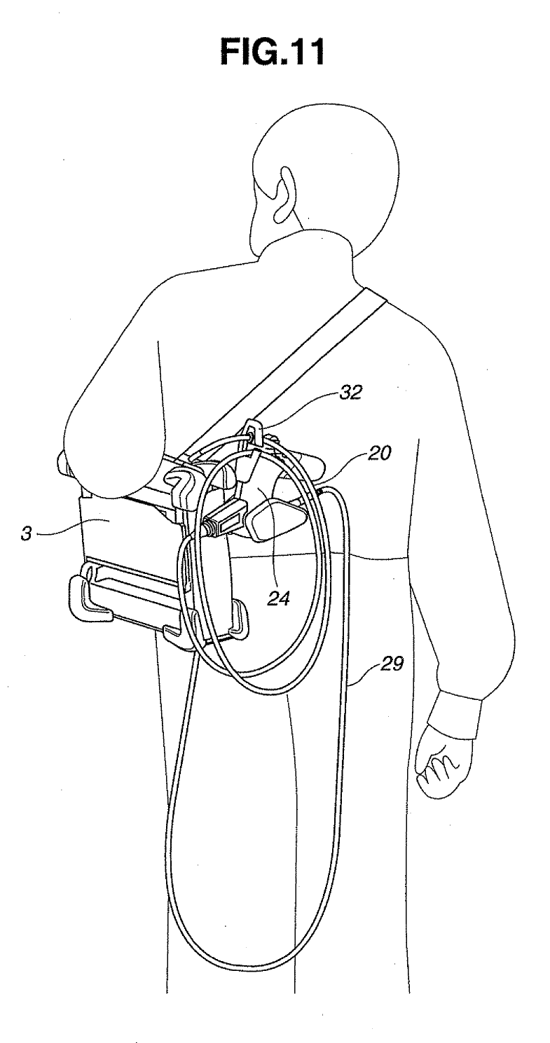

Next, there will be described an endoscope having a holding portion in which the insertion portion 20 is wound and held. An endoscope of an embodiment, as shown in FIG. 10 and FIG. 11, is configured such that the apparatus main body 3 to which the endoscope is electrically connected, and the operation portion 24 can be detachably attached to each other, and is also configured such that the thin and long flexible insertion portion 20 extended from the operation portion 24 can be wound and held in the holding portion. FIG. 10 and FIG. 11 show a form in which the apparatus main body 3 is carried in the state where the operation portion 24 is attached to the apparatus main body 3, and where the insertion portion 20 is wound and held. The endoscope according to the present embodiment is a portable type in which the insertion portion can be wound and held, when being carried. This not only makes it possible to prevent a trouble that the distal end portion and the bending portion are damaged by being accidentally subjected to a load, but also enables the endoscope to be easily carried.

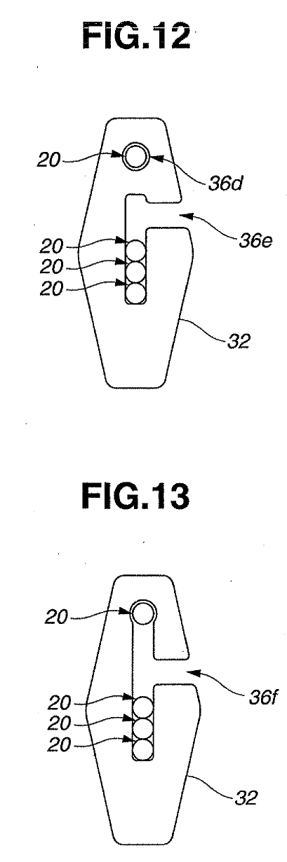

Here, as shown in FIG. 12, the holding portion in which the insertion portion 20 is wound and held, may be a recessed portion, that is, a cutout portion, which is different from a holding portion 36d for holding the distal end side portion of the insertion portion 20. Alternatively, as shown in FIG. 13, the holding portion in which the insertion portion 20 is wound and held, may be configured integrally with the holding portion 36d.

As described above, the holding member 32 also serves as the guard member for protecting the bending lever 30. Thus, the winding holding member in which the insertion portion 20 is wound and held, need not be separately provided in the operation portion 24, and hence the structure of the endoscope is simplified.

Of course, it may also be configured such that a holding portion in which the thin and long insertion portion 20 is wound and held, is arranged as a guard member for protecting the bending lever 30, and a holding portion 50, as shown in FIG. 9, only for holding the distal end portion 21 of the insertion portion 20 is also provided in the operation portion 24. In the endoscope having two or more holding portions, the form of winding the insertion portions 20 and the form of holding the distal end portion side can be changed in various ways, depending on situations, or the like, when the user moves. Thus, there is no possibility that an overload is applied to the universal cord or the distal end portion 21, and also the endoscope can be easily carried.

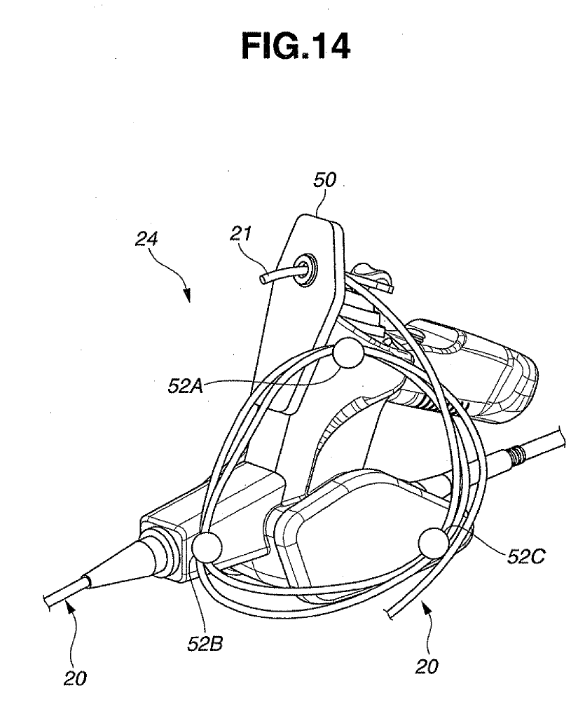

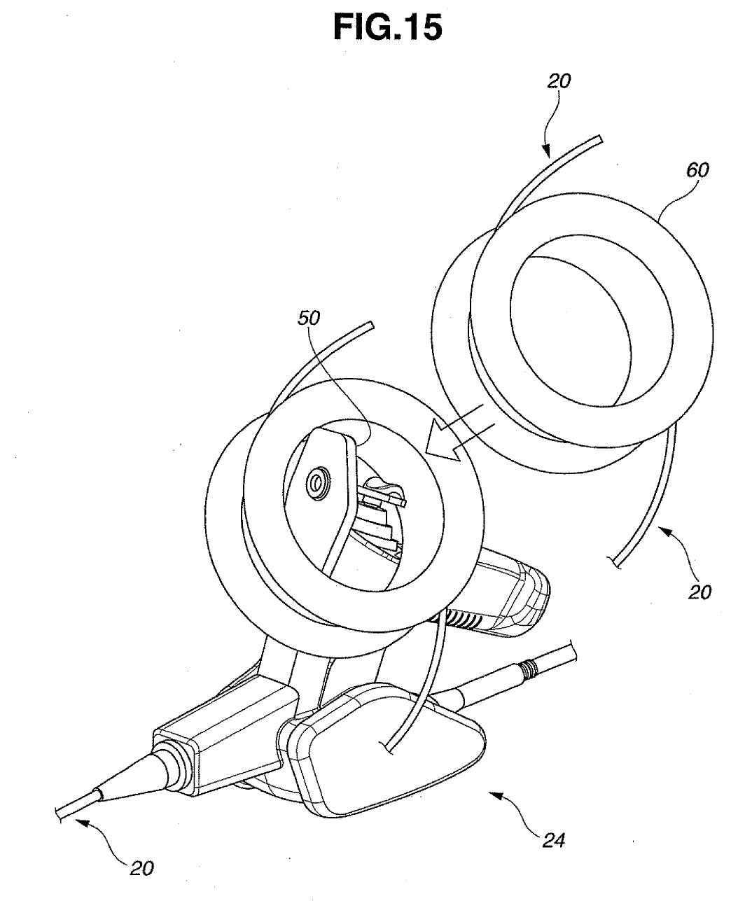

Note that the present invention is not limited to the above described winding forms of the insertion portion 20. For example, as shown in FIG. 14, three winding supporting portions 52A, 52B and 52C as supporting portions may be provided on the side surface of the operation portion 24.

Thereby, the insertion portion 20 can be wound so as to be hooked by the supporting portions on the side surface of the operation portion 24.

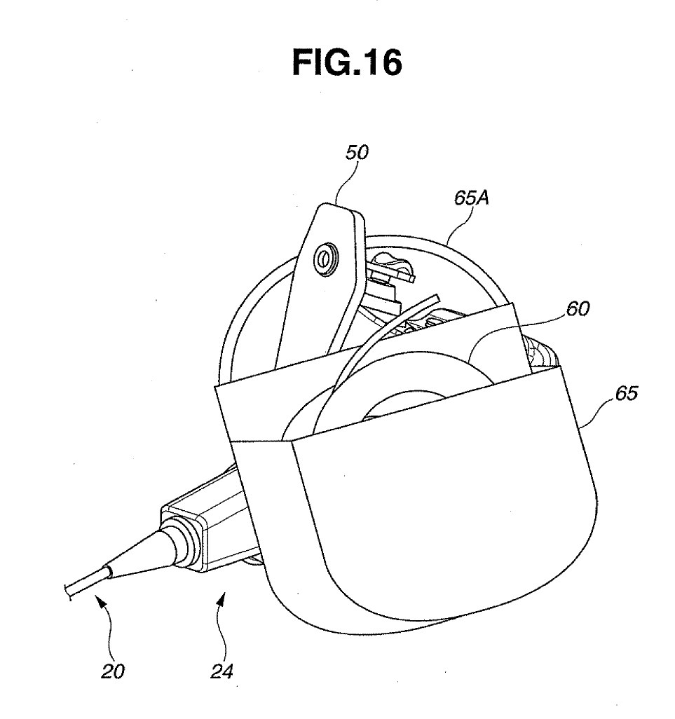

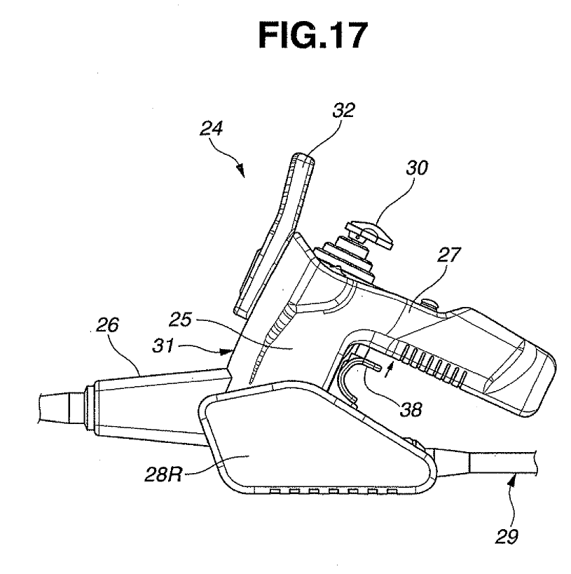

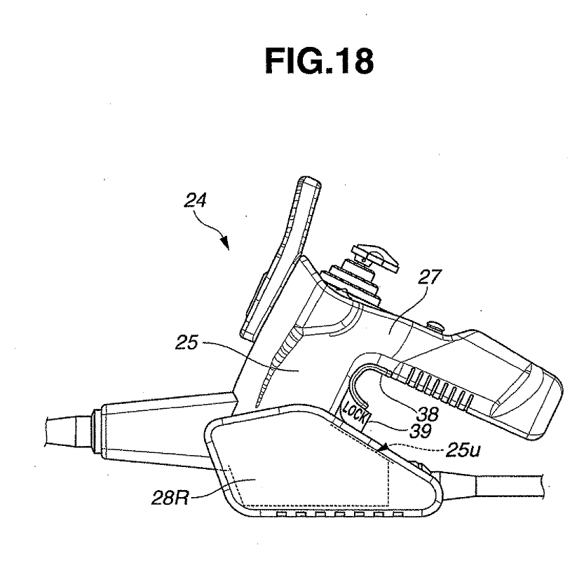

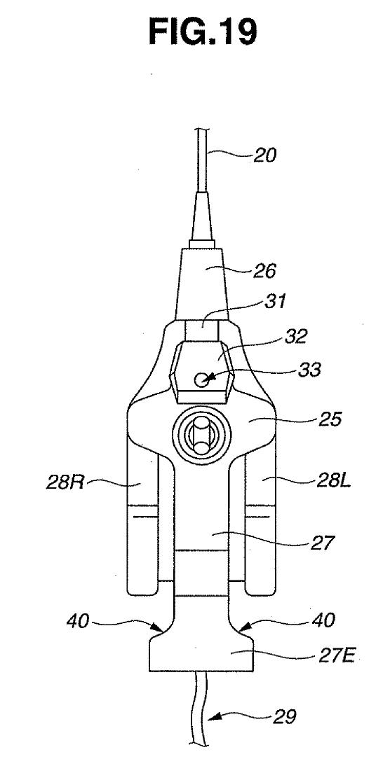

Further, even in another configuration, the insertion portion 20 can also be held by the operation portion 24. For example, as shown in FIG. 15, it may be configured such that the insertion portion 20 is wound and housed in a member in which the insertion portion 20 is wound and housed, for example, in a doughnut shaped winding portion 60 for winding the insertion portion, which winding portion is utilized when the endoscope is housed in a case, and that the member is hooked by the operation portion 24. Further, as shown in FIG. 16, it may also be configured such that the winding portion 60, in which the insertion portion 20 is wound and housed, is housed in a pocket shaped housing portion 65, and then the housing portion 65 is hooked by the operation portion 24.

In the endoscope as in the above described form, it is not necessary to wind the insertion portion 20 around the operation portion 24 each time the endoscope is taken out of the case. Thereby, the portability of the endoscope is improved.

That is, the endoscope has a housing portion 65 in which the insertion portion 20 is wound and housed, and the housing portion 65 is held by the operation portion 24. Here, the housing portion 65 is the above described housing portion 65 in which the winding portion 60 is stored. However, when only the winding portion 60 is held in the operation portion 24, the housing portion 65 means only the winding portion 60.

Note that a state where the distal end portion 21 of the insertion portion 20 is not held, is shown in FIG. 15 and FIG. 16, but of course, the distal end portion 21 of the insertion portion 20 is held in the holding portion of the holding member 50 provided in the operation portion 24. However, in the endoscope having the winding portion 60 or the housing portion 65, a holding portion (not shown) which is provided in the winding portion 60 or the housing portion 65, or which is formed by using a part of the winding portion 60 or the housing portion 65, may be used for holding the distal end portion 21 of the insertion portion 20.

Meanwhile, a bending lock knob 38 is provided on the rear face side of the operation portion main body 25 which configures the operation portion 24, as shown in FIG. 14. The bending lock knob 38 is movable in a direction of an arrow in FIG. 14, and has a function to hold the curved shape of the bending portion 22 which is provided in the insertion portion 20, in the curved state at the time when the bending lock knob 38 is moved in the direction of the arrow to reach the position shown in FIG. 15. Therefore, when the user bends the bending portion 22 into a desired shape by suitably operating the bending lever 30, the user is able to make the curved shape of the bending portion 22 held in the desired shape by moving the bending lock knob 38 to the fixed position as shown in FIG. 15. In other words, in the state where the bending lock knob 38 is arranged at the fixed position, even when the user operates the bending lever 30, the bending portion 22 is not bent, and the curved shape at the time when the bending lock knob 38 is arranged at the fixed position, is maintained.

As shown in FIG. 15, in the bending lock knob 38, there is provided a notifying portion 39 which notifies the user that the bending lock knob 38 has been moved to the fixed position. For example, the notifying portion 39 displays characters, such as “LOCK”, for notifying the user that the bending lock knob 38 is arranged at the fixed position, or is colored by a color for notifying the user that the bending lock knob 38 is arranged at the fixed position. In the present embodiment, the notifying portion 39 also serves as a finger catch preventing portion which is configured by reducing a gap between the bending lock knob 38 and the upper surface 25u of the operation portion main body 25, so as to prevent a user's finger from being inserted and caught in the gap.

Note that FIG. 14 is a view for explaining the bending lock knob provided in the operation portion. FIG. 15 is a view for explaining the effects of the notifying portion provided in the bending lock knob. FIG. 16 is a view for explaining the grasping portion proximal end portion which configures the grasping portion.

As shown in FIG. 16, the grasping portion 27 is provided with a grasping proximal end portion 27E whose external shape is changed so as to be enlarged in the right and left direction of the proximal end side. The grasping proximal end portion 27E is formed so as to project from the grasping portion 27 which is grasped by the user's fingers and palm, and has a function as a collar for preventing the grasping portion 27 from dropping from the user's hand. Further, the grasping proximal end portion 27E has a function as a supporting portion which is used when the grasping portion 27 is grasped. Thus, by making a part of the side face of the palm abutted on the side surface external shape changing portion 40 of the grasping portion proximal end portion 27E, an excellent grasping property is obtained, so as to thereby enable the grasping portion 27 to be easily grasped for a long time.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An endoscope comprising:
a thin and long flexible insertion portion;
an operation portion including a grasping portion from which the insertion portion is extended; and
a holding portion configured to hold a distal end side portion of the insertion portion provided in the operation portion.

2. The endoscope according to claim 1,
wherein the holding portion is a hole formed in the operation portion.

3. The endoscope according to claim 1,
wherein the holding portion is a hole formed in a holding member provided in the operation portion.

4. The endoscope according to claim 1,
wherein the holding portion is a recessed portion formed in a holding member provided in the operation portion.

5. The endoscope according to claim 1,
wherein the holding member is placed in the front in the insertion portion extending direction from a bending instruction mechanism provided on a top surface of the operation portion, and is provided within a range between one side surface and the other side surface of the operation portion.

6. The endoscope according to claim 1,
wherein the operation portion can be detachably attached to an apparatus main body to which the operation portion is electrically connected.

7. The endoscope according to claim 1,
wherein the insertion portion is wound and held by the holding portion.

8. The endoscope according to claim 1,
wherein the holding portion includes a supporting portion in which the insertion portion is wound and held.

9. The endoscope according to claim 1, further comprising
a housing portion in which the insertion portion is wound and held,
wherein the housing portion is held by the operation portion.

10. The endoscope according to claim 1,
wherein the holding member is a guard member configured to protect the bending instruction mechanism.

11. An endoscope comprising:
an insertion portion having a thin and long flexible bending portion;
an operation portion including a grasping portion from which the insertion portion is extended, a bending instruction mechanism configured to instruct bending of the bending portion, and a guard member configured to protect the bending instruction mechanism; and
a holding portion configured as a hole or a cutout portion formed in the guard member, and configured to hold a distal end side portion of the insertion portion.

12. The endoscope according to claim 11,
wherein the insertion portion is wound and held by the holding portion.

13. The endoscope according to claim 11,
configured as a portable type.
